(19) United States
(12) Patent Application Publication
(10) Pub. No.: US 2008/0077079 A1
(43) Pub. Date: Mar. 27, 2008

(54) Liquid Dosing Arrangement

(76) Inventor: Bruno Slettenmark, Jarfalla (SE)

Correspondence Address: SCHIFF HARDIN, LLP;PATENT DEPARTMENT, 6600 SEARS TOWER, CHICAGO, IL, 60606-6473, US

(21) Appl. No.: 11/792,748

(22) PCT Filed: Dec. 21, 2004
(86) PCT No.: PCT/IB04/52880

§ 371 Date: Jun. 8, 2007

(51) Int. Cl. A61M 5/00

(52) U.S. Cl. 604/65

(57) ABSTRACT

A liquid dosing arrangement has a liquid supply system with a first pressure sensor for measuring a first pressure p₁; a liquid delivery system, with a second pressure sensor for measuring a second pressure p₂; and an injection valve, which is arranged so that the liquid is supplied through a restricting member in the injection valve and let through the restricting member by a pulsed opening and closing valve mechanism. The pressure drop, and the dimensions of the restricting member in the injection valve, are selected to make the velocity ν of the liquid flowing through the restricting member is high enough to make the flow of liquid through the liquid delivery system independent of the viscosity of the liquid.

### BACKGROUND OF THE INVENTION

1. Field of the Invention

The present invention relates to a liquid dosing arrangement, and more specifically to an arrangement for maintaining flow accuracy without the use of a flow sensor when an injection valve is used for dosing liquids.

2. Description of the Prior Art

There is often a need for dosing liquids with good accuracy, especially in the field of medicine, e.g. during intravenous infusion or epidural anesthesia. A number of different systems exist on the market, e.g. syringe infusion pumps, peristaltic pumps and hydrostatic infusion devices. All these methods have both advantages and drawbacks with respect to for example performance, security, complexity and pricing. Syringe infusion pumps for example give good accuracy, but are quite expensive. It is therefore advantageous to instead use a simple and cheap injection valve for dosing liquid.

An injection valve releases liquid in short pulses from a pressurized liquid source. The flow of liquid can be controlled by varying the pulse frequency and/or the pulse width of the liquid pulses. The flow however varies significantly, depending on parameters such as the ambient temperature, the inner diameter of the tubing, the radius of curvature of the tubing, counter pressure from the patient, or occlusions in the catheters. The ambient temperature causes the viscosity of the liquid to change—the viscosity of water for example decreases 50% when the temperature changes from 10° C. to 40° C. The counter pressure from the patient depends on parameters such as blood pressure, and also varies due to changes in hydrostatic pressure as the patient moves. Therefore an accurate flow sensor is required in order for the flow to be controlled. Accurate flow sensors are however quite expensive and have a limited flow range, and since they are also difficult to sterilize, they are hardly suitable for the intended applications.

There is therefore a need for a method and an apparatus for using an injection valve for accurately dosing liquids without the use of an accurate flow sensor.

### SUMMARY OF THE INVENTION

To achieve this objective the present invention provides a liquid dosing arrangement having a liquid supply system in which a first pressure sensor is arranged for measuring a first pressure p₁; a liquid delivery system in which a second pressure sensor is arranged for measuring a second pressure p₂; and an injection valve, located between the liquid supply system and the liquid delivery system, the liquid being supplied through a restricting member of the injection valve, and being let through the restricting member by a pulsed opening and closing valve mechanism with opening pulses having frequency fₒ and width tₒ, and the pressure drop over the injection valve being Δp=p₁−p₂; characterized in that the pressure drop Δp, and the dimensions of the restricting member in the injection valve, are selected to make the velocity ν of the liquid flowing through the restricting member high enough to make the flow Φ of liquid through the liquid delivery system essentially independent of the viscosity of the liquid.

The invention thereby makes it possible, with a comparatively simple and inexpensive device, to achieve an accurate flow control irrespective of downstream counter pressure as well as variations in temperature and geometric variations of the tubing, and without the use of accurate flow sensors.

In a preferred embodiment, the restricting member in the injection valve is an orifice in an orifice plate. The diameter of this orifice is preferably less than 300 μm. If a higher flow rate is desirable several orifices may be used, each with a diameter of typically less than 300 μm.

The liquid dosing arrangement preferably further comprises an expansion chamber downstream of the injection valve, preferably located immediately downstream of the injection valve.

A low pass filter can be arranged to filter the signal from the second pressure sensor. In a preferred embodiment this signal is then fed to a pressure regulating system which is arranged to feed a regulating signal to a pressure regulator in order to regulate the first pressure p₁ in the liquid supply system.

The liquid dosing arrangement preferably further has an alarm which is arranged to be activated when the second pressure p₂ measured by the second pressure sensor exceeds a certain preset value pₘₐₓ, or when the flow measured by a simple monitoring flow sensor in the liquid supply system deviates beyond certain preset values.

The present invention further provides an apparatus for intravenous infusion comprising a liquid dosing arrangement according to the invention.

The present invention also provides a method of dosing liquid including the steps of providing a liquid dosing arrangement comprising a liquid supply system, a liquid delivery system and an injection valve; measuring a first pressure p₁ in the liquid supply system by the use of a first pressure sensor; and measuring a second pressure p₂ in the liquid delivery system by the use of a second pressure sensor; characterized in selecting the pressure drop Δp=p₁−p₂ over the injection valve, and the dimensions of the restricting member in the injection valve, so that the velocity ν of the liquid flowing through the restricting member becomes high enough to make the flow Φ of liquid through the liquid delivery system essentially independent of the viscosity of the liquid.

The method of dosing liquid according to the invention preferably further includes the step of providing an expansion chamber downstream of the injection valve, preferably immediately downstream of the injection valve.

In a preferred embodiment, the method further includes the steps of filtering the signal from the second pressure sensor in a low pass filter; feeding the signals from the first and second pressure sensors to a pressure regulating system; and feeding the output of the regulating system as a regulating signal to a pressure regulator in order to regulate the first pressure p₁ in the liquid supply system.

The method preferably further includes the step of activating an alarm when the second pressure p₂ measured by the second pressure sensor exceeds a certain preset value pₘₐₓ, or when the flow measured by a simple monitoring flow sensor in the liquid supply system deviates beyond certain preset values.

### DESCRIPTION OF THE DRAWINGS

FIG. 1 schematically illustrates a liquid dosing system comprising an injection valve according to one embodiment of the present invention.

FIG. 2a schematically illustrates an orifice plate which is a part of the injection valve in FIG. 1.

FIG. 2b is a side view of the orifice plate in FIG. 2a.

### DESCRIPTION OF THE PREFERRED EMBODIMENTS

The pressure Δp over an injection valve depends on the density and the viscosity of the liquid flowing through the valve as follows:

$$Δ p = {k}_{1} * \frac{ρ * {v}^{2}}{2} + {k}_{2} * η ( T ) * v$$

ν=the velocity of the liquid flowing through the restricting member
ρ=the density of the liquid (which depends very little on the temperature)
η(T)=the dynamic viscosity of the liquid (which depends very strongly on the temperature)
k₁, k₂=constants (k₁ is normally about 1-1.5, assuming that the kinetic energy is lost in eddies downstream the abrupt restriction)

For values of ν which are high enough, the viscosity part becomes negligible, since it only depends on ν, while the kinetic pressure drop depends on ν². For values of ν which are high enough, the pressure drop Δp thus becomes virtually independent of the viscosity η(T), and accordingly also of the temperature T.

For high enough values of ν we therefore have:

$$Δ p = {k}_{1} * \frac{ρ * {v}^{2}}{2} ⇒ \\ v = \sqrt{\frac{2 * Δ p}{{k}_{1} * ρ}}$$

The flow Φ through an injection valve depends on the velocity ν of the liquid and the diameter d₁ of the restricting member in the following way:

$$\begin{matrix}Φ = \frac{π * {d}_{1}^{2}}{4} * \\ v = \frac{π * {d}_{1}^{2} * \sqrt{2}}{4 * \sqrt{{k}_{1}}} * \sqrt{\frac{Δ p}{ρ}}\end{matrix}$$

Since the density ρ of the liquid is almost constant regardless of the temperature, Φ thus becomes proportional to \sqrt{Δp}, which can be kept constant by a very simple form of pressure regulation. For high enough values of ν, Φ is therefore independent of all temperature and time dependent pressure drops downstream of the injection valve. Also, the fact that Φ is proportional to \sqrt{Δp} results in that a regulating error in Δp of x % only leads to an error in Φ of x/2%.

A high velocity ν of the liquid through the restricting member in the injection valve is accomplished by letting the restricting member have a small diameter, and letting Δp be high. Δp should therefore be regulated to a value high enough, and the diameter of the restricting member selected to be small, with the additional condition that a desired maximum flow Φₘₐₓ can be accomplished and that certain practical additional conditions are met, such as the limitation that Δp can never be higher than the available pressure pᵢₙ of the employed pressure source.

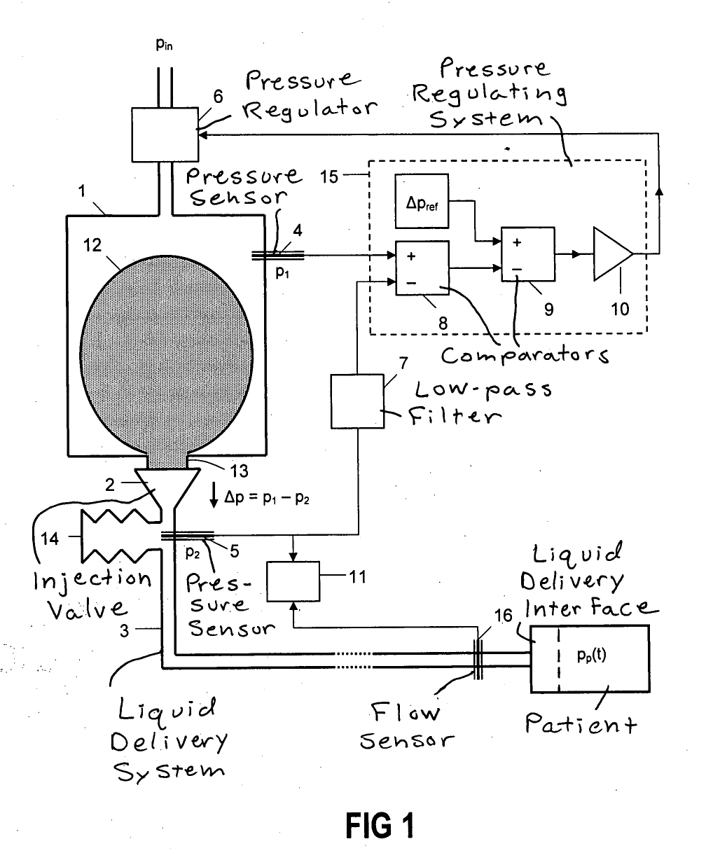

The practical application of this will now be described with reference to FIG. 1. FIG. 1 shows a liquid dosing system having a pressurized liquid source 1, an injection valve 2 and a liquid delivery system 3. The liquid delivery system 3 communicates, via a suitable liquid delivery interface with a schematically indicated patient to deliver liquid to the patient which, in the patient exhibits pressure Pp(t). The pressurized liquid source 1 can for example be an elastic container 12 under pressure in the form of a so called bag-in-bottle. The source of the driving pressure pᵢₙ can for example be the compressed air that is usually available from tapping sources in the walls in hospitals, but of course any suitable pressure source can be used. The driving pressure pᵢₙ is in hospital environments usually about 2-8 Bar. It is possible to use other methods of liquid feeding, such as mechanical pumps or other types of mechanical pressurizing means, instead of a bag-in-bottle. However, the described bag-in-bottle type liquid source is preferred due to its simplicity and to the ease of maintaining the sterility of the liquid in critical applications.

The injection valve 2 releases liquid in pulses having frequency fₒ and width tₒ from the pressurized liquid source 1. The pressure drop over the injection valve 2 is Δp=p₁−p₂. If the driving pressure pᵢₙ is constant and no regulation is effected, the pressure p₂ varies significantly, depending on parameters such as the ambient temperature, the radius of curvature of the tubing, counter pressure from the patient, or occlusions in the catheters. This results in an undesirable strong variation of the flow Φ, since this as explained above depends on the pressure drop Δp.

According to the invention the liquid dosing system therefore further has pressure sensors 4 and 5. Pressure sensor 4 measures the pressure p₁ in the liquid dosing system 1, and pressure sensor 5 measures the pressure p₂ immediately after the injection valve 2. In the configuration shown in FIG. 1 the bag 12 must be very pliable and its wall material not stretched in order for the pressure sensor 4 to measure the correct liquid pressure inside the liquid container. The signal from pressure sensor 5 is filtered through a low pass filter 7, and then fed to a pressure regulating system 15 together with the signal from pressure sensor 4. The pressure regulating system 15 outputs a regulation signal to a pressure regulator 6 which regulates the pressure p₁ in order to keep Δp=p₁−p₂ constant and equal to a certain preset value Δp_{ref}. Δp_{ref} is preferably selected to the highest possible value which is practical in the chosen application. In a hospital environment a suitable Δp_{ref} can for example be 3 Bar.

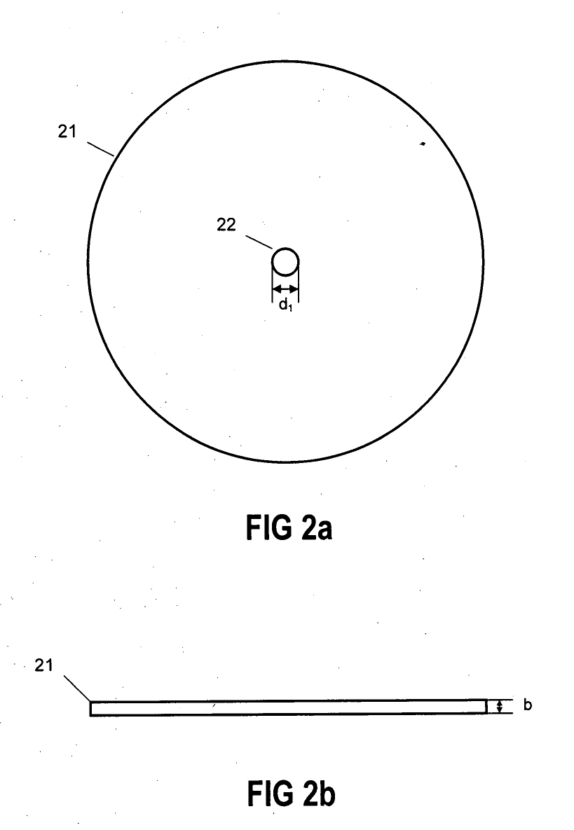

The injection valve 2 preferably has a restricting member in the form of at least one circular hole 22 drilled in an orifice plate 21, as shown in FIG. 2a, but other restricting members can also be used. The restricting member should be “short”, which is accomplished by the orifice plate 21 being thin, i.e. the thickness b in FIG. 2b being small. A thin restricting orifice plate 21 also reduces any contribution to the pressure Δp from the viscosity part. The diameter d₁ of the hole(s) 22 in the orifice plate 21 should be, as explained above, as small as possible, with respect to parameters such as Δp, the desired maximum flow Φₘₐₓ, the characteristics of the liquid, and other practical considerations. The hole(s) 22 can of course have any suitable shape, not necessarily circular. The flow Φ is accomplished by opening the injection valve 2 fully during short pulses, having frequency fₒ and width tₒ. Variation of the flow is accomplished by varying either tₒ or fₒ, or alternatively a combination of both.

The liquids used in hospitals are often water-based diluted solutions. Within the limitations explained above the velocity ν of the liquid will be the same for the same pressure drop Δp_{ref}, frequency fₒ and width tₒ. If for a specific type of injection valve the thickness b of the orifice plate is about 0.2 mm, Δp_{ref} is set to 3 Bar and the valve is opened for 2 ms every 10 seconds, the velocity ν will be 20 m/s. If the injection valve has one hole having a diameter d₁ of 100 μm, Φₘₐₓ will then be about 160 μl/s and Φₘᵢₙ will be about 0.03 μl/s.

If the diameter d₁ of the hole is only 50 μm, Φₘₐₓ will only be about 40 μl/s and Φₘᵢₙ will be about 0.01 μl/s.

If the diameter d₁ of the hole is 200 μm, Φₘₐₓ will be about 630 μl/s and Φₘᵢₙ will be about 0.13 μl/s.

If the diameter d₁ of the hole is 300 μm, Φₘₐₓ will be about 1400 μl/s and Φₘᵢₙ will be about 0.28 μl/s.

If the injection valve instead has four holes, each having a diameter d₁ of 100 μm, Φₘₐₓ will be about 630 μl/s and Φₘᵢₙ will be about 0.13 μl/s.

These values are of course just examples of working embodiments, and they are in no way limiting to the scope of the invention. There are many different types of injection valves and they all have specific characteristics, for example concerning the amount of viscous pressure drop in the valve mechanism, and the type of liquid used also affects the flow, so in practice an empiric optimization of the relevant parameters must be done, based on the above explained principles.

It is also possible to use an injection valve having an orifice plate with a hole having a diameter d₁ which is larger than 300 μm, but with a larger diameter the advantages of the claimed solution become less apparent.

A problem with liquid delivery systems such as shown in FIG. 1 is that they normally employ long plastic tubing with a small inner diameter and rigid walls. This causes them to have a very high analog “inductance”, i.e. “resistance” to quick flow changes.

The pressure drop p₂ over the tubing is a function of the flow change according to:

$$\begin{matrix}{p}_{2} = \dot{Φ} * L where \\ L = \frac{4 * ρ * l}{π * {d}_{2}^{2}}\end{matrix}$$

d₂=the inner diameter of the tubing
l=the length of the tubing
ρ=the density of the liquid

If the pulse time to is short, this results in that virtually no liquid flow at all has time to occur, and Φ thus does not become proportional to tₒ when the pulse time is increased.

One way of counteracting this effect is to place an expansion chamber 14 immediately downstream of the valve 2, with a compliance of C=ΔV/Δp, adapted so that the pressure increase Δp becomes sufficiently small for the volume ΔV that is obtained at the longest time tₒ that will be used in the pulse train. An expansion chamber 14 with a suitable shape and adequate compliance (caused by e.g. a spring load or the elasticity of the chamber itself) is therefore shown in FIG. 1 positioned immediately downstream of the injection valve 2, in order to absorb the pressure transients from the pulsed operation. Now, the effect of the inertia of the liquid in the tubing is eliminated and the mean flow through the tubing will be essentially proportional to the pulse width. Any remaining ripple in the pressure signal p₂ is filtered away in the low pass filter 7, which should have a long time constant (typically 1-10 s).

Viscous pressure drop or kinetic pressure drop between the container 12 containing the liquid and the injection valve 2 is also undesirable. Therefore the tubing 13 from the container 12 to the injection valve 2 should be as short as possible and have a sufficiently large inner diameter. Otherwise the pressure regulator 6 cannot manage to keep Δp at a constant value.

Pressure sensors 4 and 5 are preferably of a type that is simple, inexpensive and easy to clean. They may also be of a disposable type.

The pressure regulating system 15 can be designed in any suitable way, but preferably has a comparator 8 which calculates the pressure drop Δp, and another comparator 9 which compares the pressure drop Δp with the preset value Δp_{ref}. It is advantageous to also include an amplifying device 10 for amplifying the error signal before feeding it to the pressure regulator 6.

In a preferred embodiment, the liquid dosing arrangement further has an occlusion alarm 11, which is activated when p₂ exceeds a certain preset value pₘₐₓ. The occlusion alarm 11 can be used to interrupt the flow by lowering p₁ to zero, discontinue the pulsing of the valves, etc.

When a liquid dosing arrangement according to the invention is used for infusion, the pressure sensor 5 and the occlusion alarm 11 are already present in the system, since this is a requirement for infusion. The pressure sensor 5 is normally placed immediately after the valve.

A suitable flow alarm 11 at instances such as major valve leakage or errors in the regulation, which causes the flow measured by a flow sensor 16 in the liquid supply system 3 to deviate beyond certain preset values, can also be required for certain applications. In this case, a very simple type of flow sensor 16 of pressure drop type (with lesser accuracy) can be used for this function. The alarm 11 can be the same alarm as used for the occlusion alarm, or be a separate device.

Although modifications and changes may be suggested by those skilled in the art, it is the invention of the inventor to embody within the patent warranted heron all changes and modifications as reasonably and properly come within the scope of his contribution to the art.

I claim as my invention:

1. A liquid dosing arrangement, comprising:
a liquid supply system comprising a first pressure sensor that measures a first pressure p₁;
a liquid delivery system comprising a second pressure sensor that measures a second pressure p₂; and
an injection valve between the liquid supply system and the liquid delivery system comprising a restricting member through which said liquid flows, and a pulsed opening and closing valve mechanism that operates said restricting member with opening pulses having frequency fₒ and width tₒ, wherein the pressure drop Δp over the injection valve (2) is Δp=p₁−p₂;
with the pressure drop Δp, and the dimensions (b, d₁) of the restricting member in the injection valve making the velocity ν of the liquid flowing through the restricting member sufficiently high to make the flow Φ of liquid through the liquid delivery system essentially independent of the viscosity of the liquid.

2. The liquid dosing arrangement according to claim 1, wherein the restricting member in the injection valve is formed by at least one orifice in an orifice plate.

3. The liquid dosing arrangement according to claim 2, wherein the restricting member in the injection valve is formed by a plurality of orifices in an orifice plate.

4. The liquid dosing arrangement according to claim 2, wherein the at least one orifice (22) is approximately circular, and has a diameter d₁ of less than 300 μm.

5. The liquid dosing arrangement according to claim 1 comprising an expansion chamber downstream of the injection valve.

6. The liquid dosing arrangement according to claim 1, further comprising a pressure regulating system supplied with respective signals from the first and second pressure sensors, said pressure regulating system generating a regulating signal therefrom, and a pressure regulator supplied with said regulating signal that regulates the first pressure p₁ in the liquid supply system to keep Δp constant.

7. The liquid dosing arrangement according to claim 1, further comprising a low pass filter arranged to filter the signal from the second pressure sensor.

8. The liquid dosing arrangement according to claim 6, wherein the pressure regulating system comprises a first comparator device, to which the respective signals from the pressure sensors are fed, and which calculates the pressure drop Δp, and a second comparator device, which compares the pressure drop Δp, generated by the first comparator device, with a preset reference value Δp_{ref} for the pressure drop, the output from said second comparator device being the regulating signal for the pressure regulator.

9. The liquid dosing arrangement according to claim 1 comprising an alarm that is activated when the second pressure p₂ measured by the second pressure sensor exceeds a preset value pₘₐₓ.

10. The liquid dosing arrangement according to claim 1 comprising a flow sensor in the liquid supply system and an alarm that is activated when a flow measured by the flow sensor deviates beyond preset values.

11. (canceled)

12. A method of dosing liquid in a liquid dosing arrangement comprising a liquid supply system, a liquid delivery system, and an injection valve between the liquid supply system and the liquid delivery system, the method comprising the steps of:
measuring a first pressure p₁ in the liquid supply system with a first pressure sensor;
measuring a second pressure p₂ in the liquid delivery system with a second pressure sensor; and
supplying liquid through a restricting member in the injection valve by a pulsed opening and closing valve mechanism with opening pulses having frequency fₒ and width tₒ, the pressure drop Δp over the injection valve being Δp=p₁−P₂; and
selecting the pressure drop Δp=p₁−p₂ over the injection valve, and the dimensions (b, d₁) of the restricting member in the injection valve, so that the velocity ν of the liquid flowing through the restricting member insufficiently high to make the flow Φ of liquid through the liquid delivery system essentially independent of the viscosity of the liquid.

13. The method of dosing liquid according to claim 12, comprising providing an expansion chamber downstream of the injection valve.

14. The method of dosing liquid according to claim 12 comprising filtering a signal from the second pressure sensor in a low pass filter.

15. The method of dosing liquid according to claim 12 comprising the steps of:
feeding respective signals from the first and second pressure sensors to a pressure regulating system; and
feeding an output of the regulating system as a regulating signal to a pressure regulator that regulates the first pressure p₁ in the liquid supply system to keep Δp constant.

16. The method of dosing liquid according to claim 12 comprising activating an alarm when the second pressure p₂ measured by the second pressure sensor exceeds a preset value pₘₐₓ.

17. The method of dosing liquid according to claim 12 comprising activating an alarm when the flow measured by a flow sensor in the liquid supply system deviates beyond preset values.

18. An apparatus for intravenous infusion comprising
a liquid dosing arrangement comprising a liquid supply system comprising a first pressure sensor that measures a first pressure p₁, a liquid delivery system comprising a second pressure sensor that measures a second pressure p₂, and an injection valve between the liquid supply system and the liquid delivery system comprising a restricting member through which said liquid flows, and a pulsed opening and closing valve mechanism that operates said restricting member with opening pulses having frequency fₒ and width tₒ, wherein the pressure drop Δp over the injection valve (2) is Δp=p₁−p₂; and
a liquid delivery interface connected to said liquid delivery system and configured to interact with a patient to deliver said liquid to the patient.
